SEQUENCE LISTING (1 sequence)

SEQ ID NO: 1 - DNA, 28 bp, synthetic construct
  source 1..28: mol_type = other DNA
tggtggtggttgttgtggtggtggtggt

(19) United States
(12) Patent Application Publication
(10) Pub. No.: US 2024/0092813 A1
(43) Pub. Date: Mar. 21, 2024

(54) AS1411 OLIGONUCLEOTIDE COMPOSITE RUTHENIUM COMPLEX NANOPROBE, AND PREPARATION METHOD AND USE THEREOF

(71) Applicant: Guangdong Pharmaceutical University, Guangzhou, Guangdong (CN)

(72) Inventors: Wenjie MEI, Guangzhou, Guangdong (CN); Qiong WU, Guangzhou, Guangdong (CN)

(21) Appl. No.: 18/335,773

(22) Filed: Jun. 15, 2023

(30) Foreign Application Priority Data
Jun. 16, 2022 (CN) 202210682828.4

(51) Int. Cl. C07F 15/00; A61P 31/00

(52) U.S. Cl. CPC C07F 15/0053; A61P 31/00

(57) ABSTRACT

The present disclosure provides an AS1411 oligonucleotide composite ruthenium complex nanoprobe, and a preparation method and use thereof, and belongs to the technical field of nanoprobes. In the present disclosure, the AS1411 oligonucleotide having the G-quadruplex conformation has a stable structure and can specifically bind with a nucleolin on the membrane of a tumor cell; and RuPEP, the ruthenium complex having the structure of formula 1, has excellent luminescence performance and can be used as a phosphorescent probe to highlight the tumor cell. The AS1411 oligonucleotide composite ruthenium complex nanoprobe provided by the present disclosure has a good property of entering a cancer cell, and can be used as a cancer diagnosis reagent to selectively identify and activate the transportation of an NCL receptor on a surface of the cancer cell to a nucleus, thereby promoting the imaging of the cancer cell.

### CROSS-REFERENCE TO RELATED APPLICATION

This application claims priority from the Chinese patent application 202210682828.4 filed Jun. 16, 2022, the content of which is incorporated herein in the entirety by reference.

### INCORPORATION-BY-REFERENCE OF SEQUENCE LISTING OR TABLE

The material in the accompanying sequence listing is hereby incorporated by reference in its entirety into this application. The accompanying file, named “143AY_013USU_Seq_Listing.xml” was created on Jun. 13, 2023, and is 3.66 MB.

### TECHNICAL FIELD

The present disclosure relates to the technical field of the nanoprobe, and in particular to an AS1411 oligonucleotide composite ruthenium complex nanoprobe, a preparation method and use thereof.

### BACKGROUND

In current era of precision medicine and individualized medicine, molecular imaging has received much attention because of its potential utility in early confirmed diagnosis and staging of tumors, and in guidance of planning and predicting and evaluating therapeutic effects. Because of its high specificity for small molecules, an artificially synthesized short single-stranded oligonucleotide aptamer derived from SELEX (systematic evolution of ligands by exponential enrichment) has received more and more attention because of its potential utility in constructing nanoprobes. Generally, the aptamer can be bound to nanometer materials to form a nanoprobe that targets a tumor cell. For example, anti-MUC1 aptamers are loaded onto the surfaces of AuNPs through stable Au—S bonds, so as to constitute a tumor-targeting drug delivery system. Furthermore, an adenosine triphosphate (ATP)-binding aptamer can be incorporated into an DNA triangular prism to form an DNA logic device, which is expected to develop into potential applications in drug controlled release and disease treatment.

However, a probe composed of the aforementioned aptamer and nanometer materials has defects of low membrane penetration efficiency and high toxicity, which limits its further clinical application.

### SUMMARY

In view of this, an objective of the present disclosure is to provide an AS1411 oligonucleotide composite ruthenium complex nanoprobe, a preparation method and use thereof. The AS1411 oligonucleotide composite ruthenium complex nanoprobe provided by the present disclosure can be effectively absorbed and retained by a cancer cell, so as to identify the cancer cell accurately and sensitively and image the cancer cell, and has good in vivo safety.

In order to achieve the aforementioned objective of the present disclosure, the present disclosure provides the following technical solutions.

The present disclosure provides an AS1411 oligonucleotide composite ruthenium complex nanoprobe, which includes an AS1411 oligonucleotide having a G-quadruplex conformation and a ruthenium complex bound to the AS1411 oligonucleotide having the G-quadruplex conformation by hydrogen bonding, wherein the ruthenium complex has a structure of Formula 1:

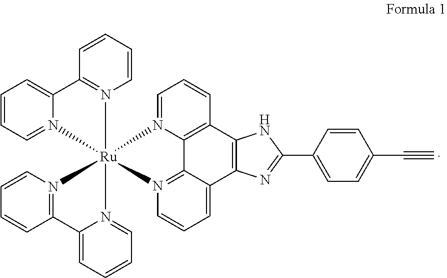

Preferably, the content of ruthenium element in the AS1411 oligonucleotide composite ruthenium complex nanoprobe is 3-10 wt %.

Preferably, the particle size of the AS1411 oligonucleotide composite ruthenium complex nanoprobe is 200-500 nm.

The present disclosure provides a preparation method of the AS1411 oligonucleotide composite ruthenium complex nanoprobe, including the following steps:
mixing a dispersion of an AS1411 oligonucleotide having a G-quadruplex conformation with a ruthenium complex having a structure of Formula 1, and performing self-assembly and dialysis, so as to obtain the AS1411 oligonucleotide composite ruthenium complex nanoprobe.

Preferably, the self-assembly is performed at a temperature of 25-40° C. for a period of 4-12 h.

Preferably, a molecular weight cutoff of the dialysis is 0.5-3.0 kDa; and the dialysis is performed at a temperature of 25-40° C. for a period of 1-3 days.

Preferably, a method for preparing the AS1411 oligonucleotide having the G-quadruplex conformation includes the following steps:
mixing an AS1411 oligonucleotide with a buffer solution containing potassium ion, and sequentially performing high temperature denaturation and low temperature renaturation, so as to obtain the AS1411 oligonucleotide having the G-quadruplex conformation;
wherein the high temperature denaturation is performed at a temperature of 90-100° C. for a period of 5 min; and the low temperature renaturation is performed at a temperature of 4-8° C. for a period of 24-72 h.

The present disclosure provides use of the AS1411 oligonucleotide composite ruthenium complex nanoprobe in preparation of a cancer diagnostic reagent.

Preferably, the cancer diagnostic reagent is a diagnostic reagent for breast cancer.

The present disclosure provides an AS1411 oligonucleotide composite ruthenium complex nanoprobe (abbreviated as AS1411@RuPEP), which includes an AS1411 oligonucleotide having a G-quadruplex conformation and a ruthenium complex bound to the AS1411 oligonucleotide having the G-quadruplex conformation by hydrogen bonding, wherein the ruthenium complex has a structure of Formula 1. In the present disclosure, the AS1411 oligonucleotide having the G-quadruplex conformation has a stable structure and can specifically bind with a nucleolin (NCL) on the tumor cell membrane; and RuPEP, the ruthenium complex having the structure of Formula 1, has excellent luminescence property and can be used as a phosphorescent probe to highlight tumor cells. In the present disclosure, the AS1411 oligonucleotide having the G-quadruplex conformation has a folded and stacked quadruplex helical structure, which can bind with the ruthenium complex RuPEP in a groove manner, thereby inducing self-assembly of AS1411 to form a nanoprobe. Meanwhile, the N atom on an imidazole ring of the ruthenium complex RuPEP can form two intramolecular hydrogen bonds with two H atoms on G15 and T16 residues in the AS1411 oligonucleotide having the G-quadruplex conformation, which improves the binding stability. The AS1411 oligonucleotide composite ruthenium complex nanoprobe provided by the present disclosure has a good property of entering a cancer cell, and thus can be used as a cancer diagnostic reagent to selectively identify and activate the transportation of an NCL receptor on a surface of the cancer cell to a nucleus, thereby facilitating the imaging of the cancer cell. Meanwhile, the nanoprobe provided by the present disclosure has good in vivo safety.

### BRIEF DESCRIPTION OF THE DRAWINGS

FIG. 1 shows a construction process of an AS1411@RuPEP nanoprobe and a principle of using it in imaging of breast cancer by NCL-targeting identification;

FIG. 2 is a schematic diagram of hydrogen bonding between a ruthenium complex RuPEP and an AS1411 oligonucleotide having a G-quadruplex conformation;

FIG. 3 shows electron absorption spectra and fluorescence emission spectra of RuPEP and AS1411@RuPEP;

FIG. 4 is a TEM image of the AS1411@RuPEP nanoprobe;

FIG. 5 is a micrograph of the AS1411@RuPEP nanoprobe under an atomic force microscope;

FIG. 6 is an EDS analysis chart of an elemental spectrum of the AS1411@RuPEP nanoprobe;

FIG. 7 is an EDS mapping diagram of the AS1411@RuPEP nanoprobe;

FIG. 8 is a particle size distribution diagram of the AS1411@RuPEP nanoprobe;

FIG. 9 is a schematic diagram of a process of the nanoprobe entering a nucleus through endocytosis;

FIG. 10 shows the cellular localization of AS1411@RuPEP (5 μM) in an MDA-MB-231 cell;

FIG. 11 shows a real-time imaging result of MDA-MB-231 breast cancer cells treated with AS1411@RuPEP within 2 h;

FIG. 12 shows an imaging result of the probe transferred from an extracellular environment to the nucleus;

FIG. 13 shows an imaging result of the MDA-MB-231 cells treated with AS1411@RuPEP under a biological transmission electron microscope;

FIG. 14 shows a process of selective imaging of a tumor cell through targeted identification of an NCL on a cell membrane surface by the AS1411@RuPEP nanoprobe;

FIG. 15 shows distribution results of the NCL in breast cancer MDA-MB-231, MCF-7 cells and normal human MCF-10A cells;

FIG. 16 shows expression results of the NCL in breast cancer MDA-MB-231, MCF-7 cells and normal human MCF-10A cells;

FIG. 17 shows the localization of the AS411@RuPEP nanoprobe in breast cancer MDA-MB-231, MCF-7 cells and normal human MCF-10A cells;

FIG. 18 is an LSCM image of MDA-MB-231 and MCF-10A cells after being co-cultured with AS1411@RuPEP for 6 h;

FIG. 19 shows an analysis result of the superimposed data of FIG. 18;

FIG. 20 shows specific tumor-targeted images taken at different time points after injection of the nanoprobe;

FIG. 21 shows the result of quantitative determination of fluorescence intensity of AS1411@RuPEP in a tumor region and a non-tumor region of a mouse;

FIG. 22 shows the result of quantitative determination of fluorescence intensity of AS1411@RuPEP (average cps) in an anatomical organ or tissue;

FIG. 23 shows the tissue distribution and drug metabolism results of AS1411@RuPEP at 24, 72 and 108 h;

FIG. 24 shows the pathological changes of different tissues after injection of the nanoprobe through tail vein;

FIG. 25 shows the histochemical analysis of hematoxylin-eosin staining of sections of the human breast cancer tissue from samples of 5 patients with invasive ductal carcinoma;

FIG. 26 shows the imaging result of frozen sections of a fresh human invasive ductal carcinoma tissue observed under a fluorescence microscope;

FIG. 27 shows the distribution of the nucleolin and the nanoprobe in a cancer region and a paracancer region as observed by CLSM amplification;

FIG. 28 is a combined curve for analyzing the three-channel emission intensity of paracancer cells by Image-Pro Plus software;

FIG. 29 shows the quantitative analysis of NCL expression and tumor grading in the samples of the 5 cases of invasive ductal carcinoma;

FIG. 30 shows the quantitative analysis of NCL expression and tumor grading in the samples of the 5 cases of invasive ductal carcinoma;

FIG. 31 is a flowchart of operations of detecting tumor tissue samples with the nanoprobe;

FIG. 32 shows the pathological features of HE staining in normal and grade I-III tissues of invasive ductal carcinoma samples;

FIG. 33 shows the results of imaging normal and different tumor-grading samples by the AS1411@RuPEP nanoprobe;

FIG. 34 is an emission intensity curve of the nanoprobe at a white marking trace in different samples; and

FIG. 35 shows the statistical analysis result of the equal-area average intensity of duplicates of normal and different tumor-grading samples.

### DESCRIPTION OF EMBODIMENTS

The present disclosure provides an AS1411 oligonucleotide composite ruthenium complex nanoprobe, which includes an AS1411 oligonucleotide having a G-quadruplex conformation and a ruthenium complex (abbreviated as RuPEP) bound to the AS1411 oligonucleotide having the G-quadruplex conformation by hydrogen bonding, wherein the ruthenium complex RuPEP has a structure of Formula 1:

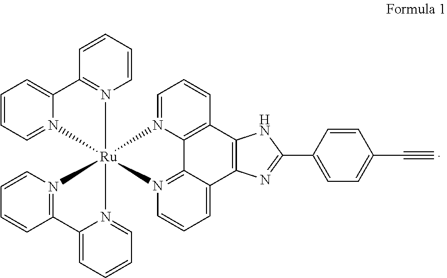

In the present disclosure, the sequence of the AS1411 oligonucleotide is as shown in SEQ ID NO: 1, which is specifically from 5′ to 3′ indicated as TGGTGGTGGTTGTTGTGGTGGTGGTGGT.

In the present disclosure, the source of the AS1411 oligonucleotide is preferably commercially available. As a specific embodiment of the present disclosure, the AS1411 oligonucleotide is purchased from Sangon Biotech (Shanghai) Co., Ltd.

The present disclosure has no special requirement on the source of the ruthenium complex RuPEP, and the ruthenium complex RuPEP having the structure of Formula 1 can be commercially available in the art or self-prepared. When self-prepared, the preparation method preferably includes the following steps.

Into a 30 mL microwave reaction tube added is [Ru(bpy)₂Cl₂] 2H₂O (105 mg, 0.2 mmol), p-EPIP (236.7 mg, 0.3 mmol), and a mixed solvent of ethylene glycol and water, introduced with argon for 10 min, and subjected to microwave-assisted heating at 120° C. for 20 min. After the reaction is completed, a mixture is cooled to room temperature, diluted by addition of water, filtered to remove insoluble substances, so as to obtain a deep red filtrate. The filtrate is added with excessive sodium perchlorate, and allowed to stand overnight to produce a large amount of orange-red precipitate. The precipitate is obtained by filtering, washed with water and ethyl ether for several times respectively, and dried in a vacuum dryer to obtain an orange-yellow solid. The crude product is dissolved in acetonitrile, passes through a 200-300 mesh neutral alumina column, washed with acetonitrile to elute a main red component, and spin-dried at reduced pressure to remove the solvent, so as to obtain a brownish red solid as the ruthenium complex RuPEP.

In the present disclosure, the content of ruthenium element in the AS1411 oligonucleotide composite ruthenium complex nanoprobe is preferably 3-5 wt %, and more preferably 4 wt %.

In the present disclosure, the particle size of the AS1411 oligonucleotide composite ruthenium complex nanoprobe is preferably 200-500 nm, and more preferably 300-400 nm.

In the present disclosure, the AS1411 oligonucleotide having the G-quadruplex conformation has a folded and stacked quadruplex helical structure, which can bind with the ruthenium complex RuPEP in a groove manner, thereby inducing self-assembly of AS1411 to form a nanoprobe. Meanwhile, the N atom on an imidazole ring of the ruthenium complex RuPEP can form two intramolecular hydrogen bonds with two H atoms on G15 and T16 residues in the AS1411 oligonucleotide having the G-quadruplex conformation, which improves the binding stability.

In the present disclosure, the AS1411 oligonucleotide having the G-quadruplex conformation can specifically bind with a nucleolin (NCL) on the tumor cell membrane for targeted identification of a cancer cell; the ruthenium complex RuPEP has excellent luminescence property, can be used as a phosphorescent probe to highlight tumor cells, and has good biological safety. The AS1411 oligonucleotide composite ruthenium complex nanoprobe provided by the present disclosure has a good property of entering a cancer cell, and thus can be used as a cancer diagnostic reagent to selectively identify and activate the transportation of an NCL receptor on a surface of the cancer cell to a nucleus, and locates the nanoprobe into the nucleus of the tumor cell through an endocytosis process, thereby facilitating the imaging of the cancer cell.

The present disclosure provides a preparation method of the AS1411 oligonucleotide composite ruthenium complex nanoprobe, including the following steps:
mixing a dispersion of an AS1411 oligonucleotide having a G-quadruplex conformation with a ruthenium complex having a structure of Formula 1, and performing self-assembly and dialysis, so as to obtain the AS1411 oligonucleotide composite ruthenium complex nanoprobe.

In the present disclosure, a dispersion of the AS1411 oligonucleotide having the G-quadruplex conformation is preferably a dispersion of the AS1411 oligonucleotide having the G-quadruplex conformation in a buffer solution containing potassium ion. In the present disclosure, the buffer solution containing potassium ion is preferably a Tris-HCl KCl buffer solution, and the pH value of the Tris-HCl KCl buffer solution is preferably 7.2. In the present disclosure, the concentration of the dispersion of the AS1411 oligonucleotide having the G-quadruplex conformation is preferably 50-100 μmol/L, and more preferably 60-80 μmol/L.

In the present disclosure, a method for preparing the AS1411 oligonucleotide having the G-quadruplex conformation preferably includes the following steps:
mixing an AS1411 oligonucleotide with a buffer solution containing potassium ion, and sequentially performing high temperature denaturation and low temperature renaturation, so as to obtain the AS1411 oligonucleotide having the G-quadruplex conformation.

In the present disclosure, the buffer solution containing potassium ion is preferably a Tris-HCl KCl buffer solution. The present disclosure has no special requirement on the mixing manner, and a mixing manner well known to those skilled in the art can be used, in particular such as mixing under stirring.

In the present disclosure, high temperature denaturation is performed at a temperature of preferably 90-100° C., and more preferably 95° C. for a period of preferably 5 min; and low temperature renaturation is performed at a temperature of preferably 4-8° C., and more preferably 5-6° C. for a period of preferably 24-72 h, and more preferably 36-60 h. In the present disclosure, the purpose of the high temperature denaturation is to denature an DNA sequence into a single-stranded form, and the purpose of the low temperature renaturation is to make the single-stranded DNA form a secondary structure of G-quadruplex DNA around K+ ions.

In the present disclosure, the ruthenium complex having the structure of Formula 1 is preferably provided in a form of a dispersion in a buffer solution. In the present disclosure, the buffer solution is preferably a Tris-HCl KCl buffer solution, and the pH value of the Tris-HCl KCl buffer solution is preferably 7.2. In the present disclosure, the concentration of the dispersion of the ruthenium complex in the buffer solution is preferably 20-100 μmol/L, and more preferably 50 μmol/L.

In the present disclosure, the molar ratio of the AS1411 having the G-quadruplex conformation to the ruthenium complex is preferably 1:1.

The present disclosure has no special requirement on the mixing manner, and a mixing manner well known to those skilled in the art can be used, in particular such as mixing under stirring.

In the present disclosure, the self-assembly is performed at a temperature of preferably 25-40° C., and more preferably 37° C. for a period of preferably 4-12 h, and more preferably 8-10 h.

In the present disclosure, the molecular weight cutoff of the dialysis is preferably 0.5-3.0 kDa, and more preferably 1-2 kDa; and in the present disclosure, the dialysis is performed at a temperature of preferably 37° C. for a period of preferably 1-3 days.

The present disclosure provides use of the AS1411 oligonucleotide composite ruthenium complex nanoprobe in preparation of a cancer diagnostic reagent. In the present disclosure, the cancer diagnostic reagent is preferably a diagnostic reagent for breast cancer, and further preferably a breast invasive ductal carcinoma diagnostic reagent.

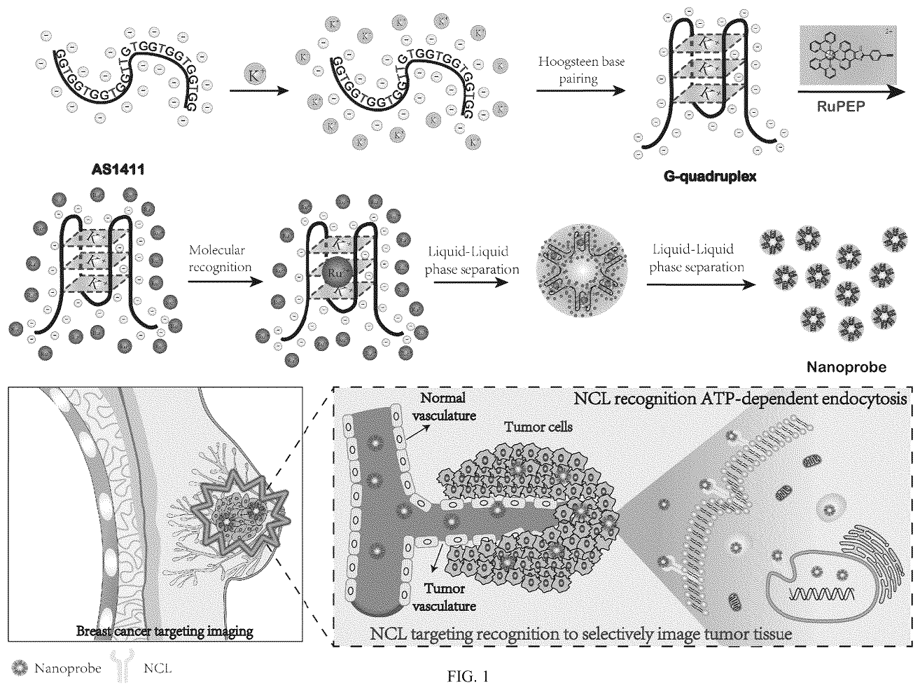

In the present disclosure, the construction process of the AS1411 @RuPEP nanoprobe and a principle of using it in imaging of breast cancer by NCL-targeting identification are shown in FIG. 1.

The AS1411 oligonucleotide composite ruthenium complex nanoprobe provided by the present disclosure, a preparation method and use thereof will be described in detail in conjunction with examples, but they should not be construed as limiting the scope of protection of the present disclosure.

### Example 1

### (1) Synthesis of Ruthenium Complex RuPEP

Into a 30 mL microwave reaction tube added was [Ru(bpy)₂Cl₂] 2H₂O (105 mg, 0.2 mmol), p-EPIP (236.7 mg, 0.3 mmol), and a mixed solvent of ethylene glycol and water, introduced with argon for 10 min, and subjected to microwave-assisted heating at 120° C. for 20 min. After the reaction was completed, the mixture was cooled to room temperature, diluted by addition of water, filtered to remove insoluble substances, so as to obtain a deep red filtrate. The filtrate was added with excessive sodium perchlorate, and allowed to stand overnight to produce a large amount of orange-red precipitate. The precipitate was obtained by filtering, washed with water and ethyl ether for several times respectively, and dried in a vacuum dryer to obtain an orange-yellow solid. The crude product is dissolved in acetonitrile, passes through a 200-300 mesh neutral alumina column, washed with acetonitrile to elute a main red component, and spin-dried at reduced pressure to remove the solvent, so as to obtain a brownish red solid as the ruthenium complex RuPEP.

### (2) Synthesis of AS1411@RuPE Nanoprobe

The used AS1411 oligonucleotide was purchased from Sangon Biotech (Shanghai) Co., Ltd., and the sequence of the AS1411 oligonucleotide was as shown in SEQ ID NO: 1, which is specifically from 5′ to 3′ indicated as TGGTGGTGGTTGTTGTGGTGGTGGTGGT.

The AS1411 oligonucleotide was mixed with a Tris-HCl KCl buffer, denatured at 95° C. for 5 minutes, and then renatured at 4° C. for 24 hours to obtain a dispersion of AS1411 having a G-quadruplex conformation with a concentration of 50 μM.

The dispersion of the AS1411 having the G-quadruplex conformation was mixed with a ruthenium complex RuPEP (50 μM, Tris-HCl KCl buffer) according to a volume ratio of 1:1, and dialyzed by using a dialysis bag with a molecular weight cutoff of 0.5-3.0 kDa at 37° C. for 3 days, and the resultant product was freeze-dried to obtain the AS1411@RuPE nanoprobe.

The N atom on the imidazole ring of the ruthenium complex RuPEP could form two intramolecular hydrogen bonds with two H atoms on G15 and T16 residues in the AS1411 oligonucleotide having the G-quadruplex conformation, and its schematic diagram was shown in FIG. 2.

### (3) Characterization of AS1411@RuPE Nanoprobe

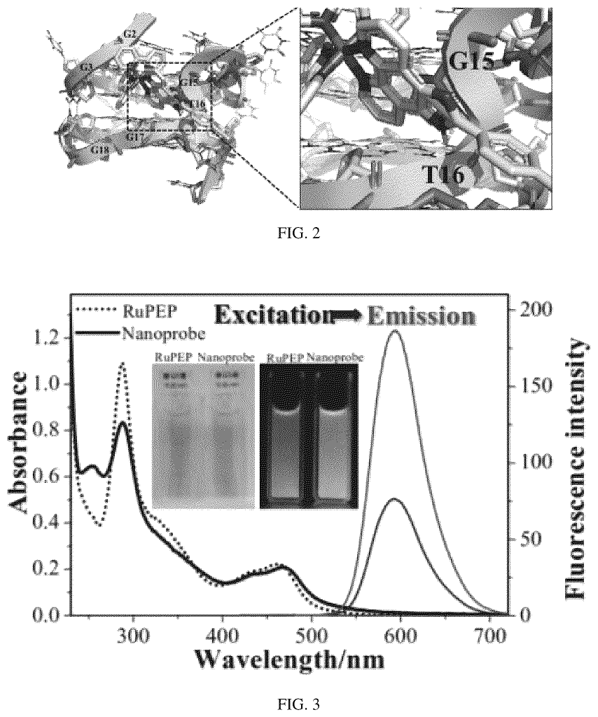

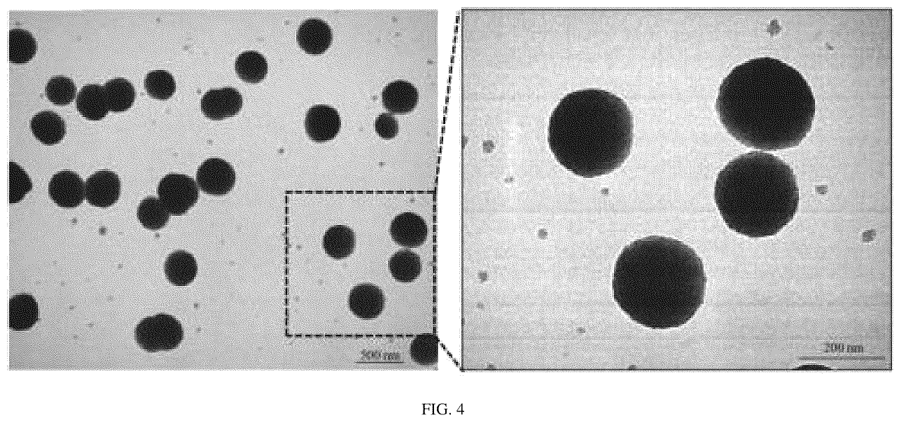

The electron absorption spectra and fluorescence emission spectra of RuPEP (5 μM) and AS1411@RuPEP (5 μM) in a PBS solution were shown in FIG. 3. As could be seen from FIG. 3, the fluorescence of the nanoprobe was stronger than that of the equimolar RuPEP, which might be attributed to the initiation of the interaction between RuPEP and AS1411, which enhanced the fluorescence emission of the nanoprobe.

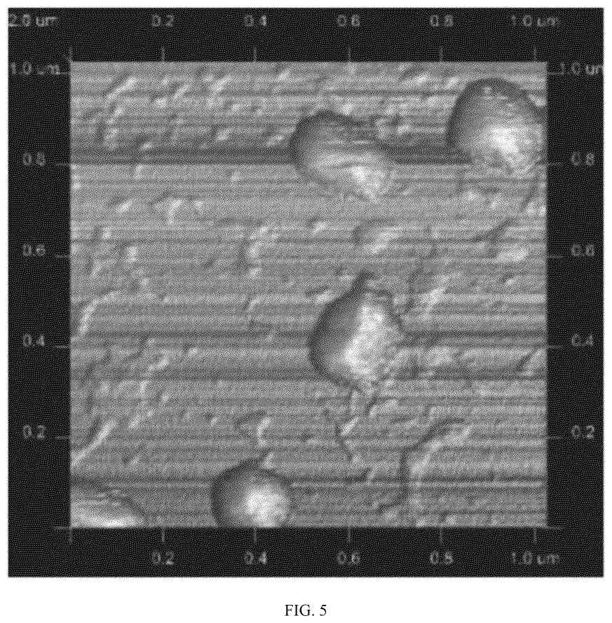

An TEM image of the AS1411@RuPEP nanoprobe was shown in FIG. 4. As could be seen from FIG. 4, the AS1411@RuPEP nanoprobe was monodispersed nanoparticles with an average diameter of 200 nm.

An atomic force micrograph (AFM) of the AS1411@RuPEP nanoprobe was shown in FIG. 5, and it could be seen from FIG. 5 that the nanoparticles of the AS1411@RuPEP nanoprobe were uniformly distributed with an average diameter of 200 nm.

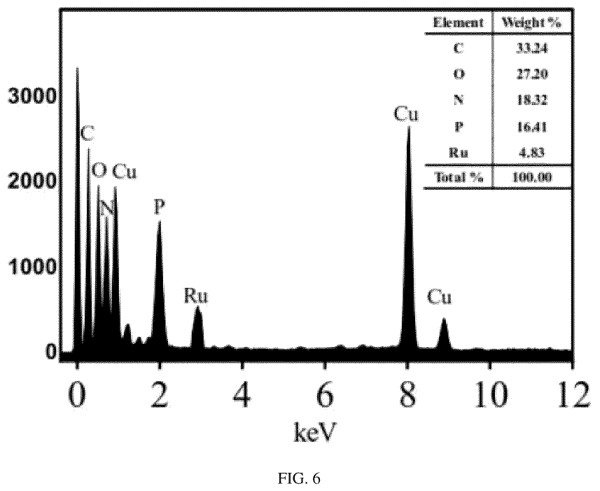

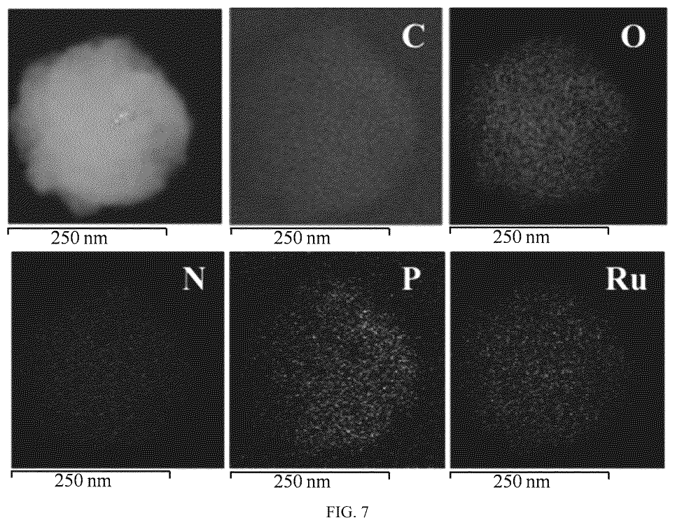

An EDS analysis diagram of an elemental spectrum of the AS1411@RuPEP nanoprobe was shown in FIG. 6. As could be seen from FIG. 6, the P atom (16.41%) in an AS1411 molecule had a strong signal, and the Ru atom (4.83%) in RuPEP had an obvious signal. Furthermore, there were also obvious signal peaks of C (33.24%), N (18.32%) and O (27.20%) in AS1411 and RuPEP. The dispersion spectrum analysis showed that the assembly of AS1411 and RuPEP had successfully constructed the nanoprobe.

The EDS mapping diagram of the AS1411@RuPEP nanoprobe was shown in FIG. 7. As could be seen from FIG. 7, the distribution of elements C, P and Ru was uneven, P and Ru were mainly concentrated in the cores of the particles, while C was preferentially found on the surfaces of the particles. We had observed similar properties throughout the sample. P and Ru had very strong spatial correlation, and these two types of atoms were abundant in the center of the particles.

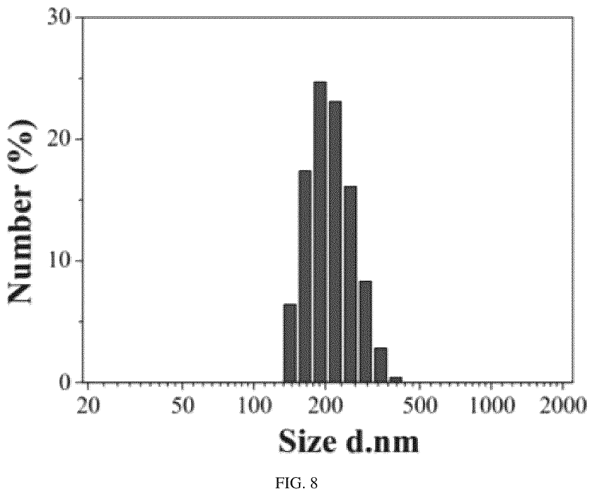

A particle size distribution diagram of the AS1411@RuPEP nanoprobe was shown in FIG. 8. The particle size distribution was measured by a DLS method. As could be seen from FIG. 8, the average length of the nanoprobe ranged from 200-500 nm.

### Example 2 Cell Uptake and Localization of Nanoprobe in Nucleus of Tumor Cell

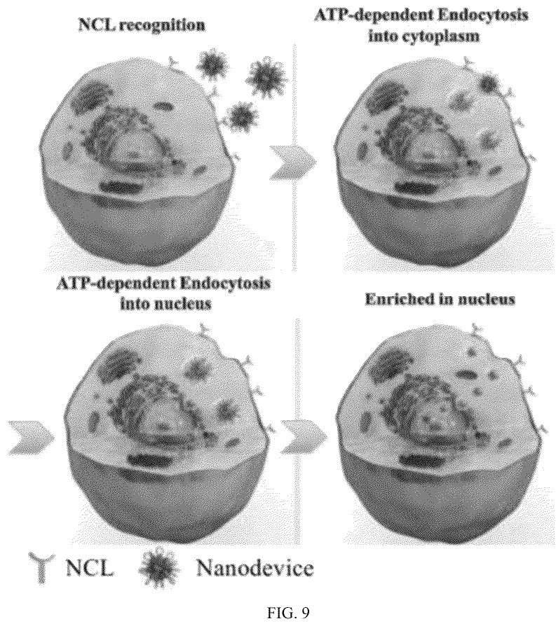

(1) The targeted identification ability of the nanoprobe to the tumor cell was studied by using a breast cancer cell MDA-MB-231 with high expression of NCL. A schematic diagram of a process of the nanoprobe entering the nucleus through endocytosis was shown in FIG. 9.

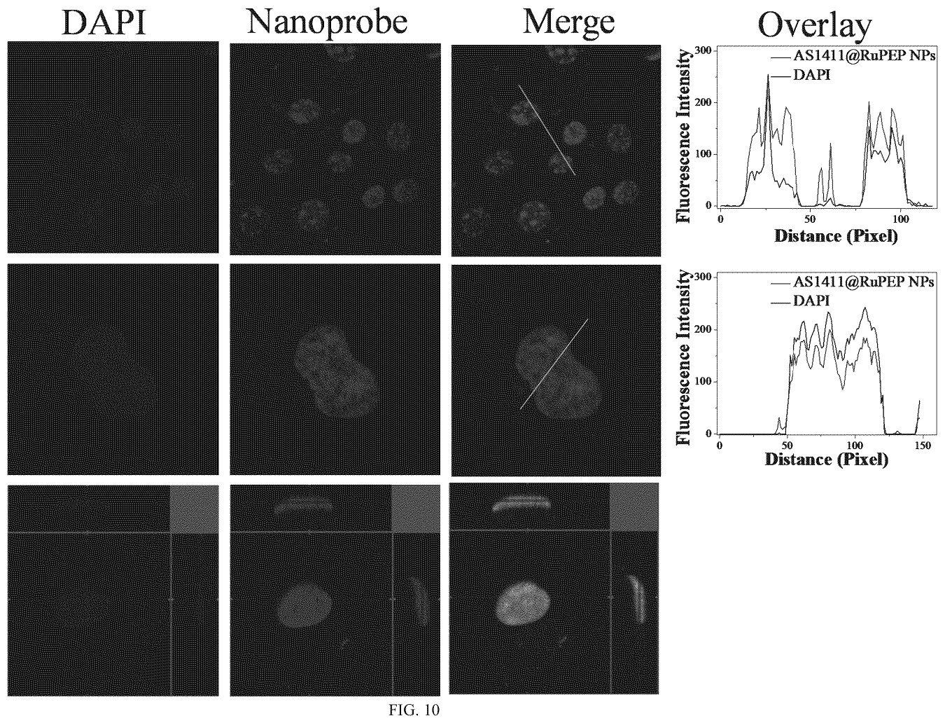

The cellular localization of AS1411@RuPEP (5 μM) in MDA-MB-231 cells was shown in FIG. 10. As could be seen from FIG. 10, after incubation with the MDA-MB-231 breast cancer cells, the nanoprobe was completely absorbed by the cells and emitted intense red phosphorescence from the nucleus. It could be observed that the red phosphorescence was co-located in the same position and completely covered the blue fluorescence band. In the enlarged image, a two-color fluorescence band was limited to the nucleus. The overlap rate of the three color bands from the nanoprobe and DAPI was very close to 100%. Moreover, the red fluorescence from 3D tomographic imaging of deep slice images filled the whole nucleus and matched the staining pattern observed from the nanoprobe and DAPI. These results showed that the nanoprobe was effectively absorbed and retained by the tumor cell and located in the nucleus.

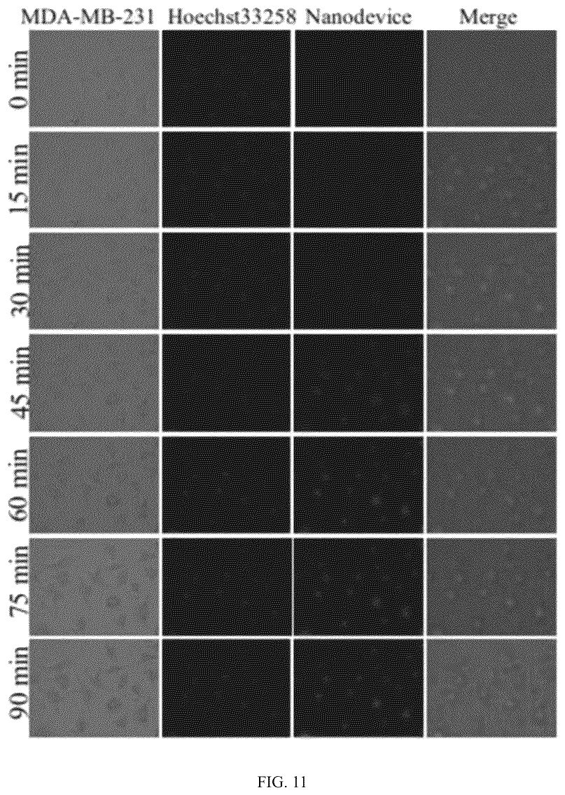

The real-time imaging results of MDA-MB-231 breast cancer cells treated with AS1411@RuPEP(5 μM) within 2 hours were shown in FIG. 11. In FIG. 11, the cell morphology was captured by a phosphorescent microscope every 15 minutes.

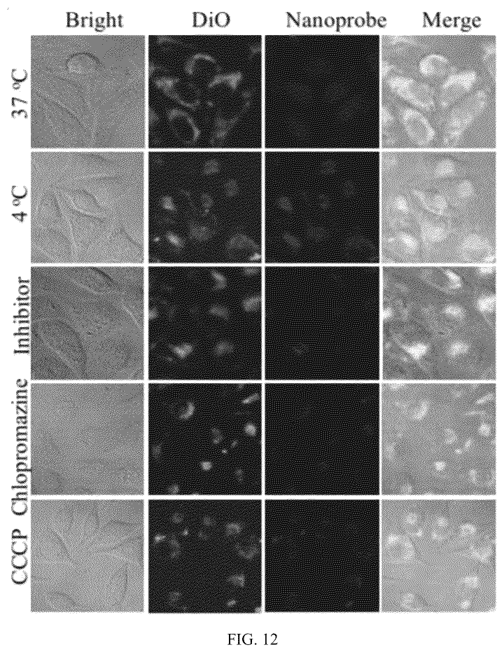

(2) In order to determine the uptake mechanism of the probe from an extracellular environment to the nucleus, MDA-MB-231 cells were cultured at 37° C. and 4° C. with the AS1411@RuPEP nanoprobe (5 μM) for 6 hours respectively. After incubation at 37° C., most of the nanoprobes were located in the nucleus, while after incubation at 4° C., the nanoprobes still remained in the cytoplasm. The results were shown in FIG. 12. Based on the results, we assumed that the nanoprobe entered the nucleus through an energy-dependent pathway, which was derived from an active transport mechanism that drove the NCL into the nucleus through intracellular trans-localization. These processes slowed down at 4° C. In general, endocytosis described a common mechanism of various extracellular substances entering a cell, which was an energy-dependent process. In this process, a pore coated with a cage shaped protein was a main plasma membrane specialized carrier that participated in the absorption of various molecules.

In order to clarify the specific endocytosis pathway of the nanoprobe involved in cell internalization, MDA-MB-231 cells were firstly pretreated with chlorpromazine (a clathrin-dependent inhibitor, 6 nM) for 1 hour before incubation with the nanoprobe. Then we observed that the fluorescence signal of the nanoprobe was mainly located on the cell membrane surface, and meanwhile there was almost no fluorescence distribution in cytoplasm. These data showed that living cancer cells treated the nanoprobe through an endocytosis pathway. It is well known that 2-deoxy-D-glucose and oligomycin, as a common combination of ionophore inhibitors, could reduce the ability of ATP synthesis, and thus were used for determining a mechanism of intrinsic nuclear aggregation. After treatment with 2-deoxy-D-glucose and oligomycin, the staining of the nucleus by the nanoprobe was significantly inhibited. This data once again supported the viewpoint that the main reason why the nanoprobe entered the nucleus was through an energy-dependent active transport pathway.

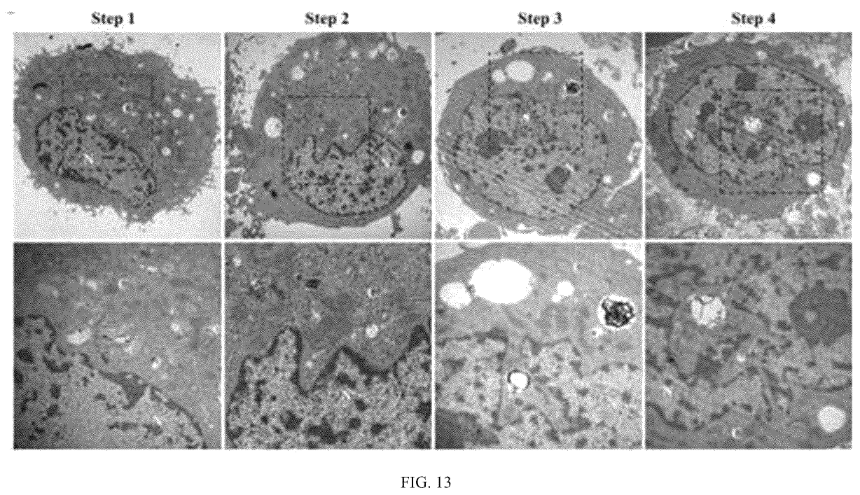

(3) At 37° C., MDA-MB-231 cells were treated with AS1411@RuPEP for 6 hours, and the imaging result of the MDA-MB-231 cells under a biological transmission electron microscope was shown in FIG. 13. As could be seen from FIG. 13, the nanoprobe was wrapped in vesicles in cytoplasm and nucleus. It could be observed that the nanoprobes could induce the MDA-MB-231 cells to produce multiple vesicles to carry them into the cytoplasm and move to the vicinity of the nuclear membrane (yellow arrow, steps 1 and 2). Many nanoprobe complexes with different sizes and shapes were found in these vesicles. These vesicles containing nanoprobe particles gradually approached the nucleus, and they contacted the nuclear membrane to cause vesicle rupture (step 2), and then the nanoprobe particles entered the nucleus through ATP-dependent endocytosis (step 3). An image of the nanoprobe complexes escaping from the vesicles was displayed by the yellow arrow in the nucleus (step 4). Escape from the vesicles was an important function for them to complete various activities. Therefore, it was assumed that the distribution of the nanoprobe particles in the nucleus was related to ATP-dependent NCL transport, which process depended on the identification and NCL binding of the AS1411 component by the nanoprobe.

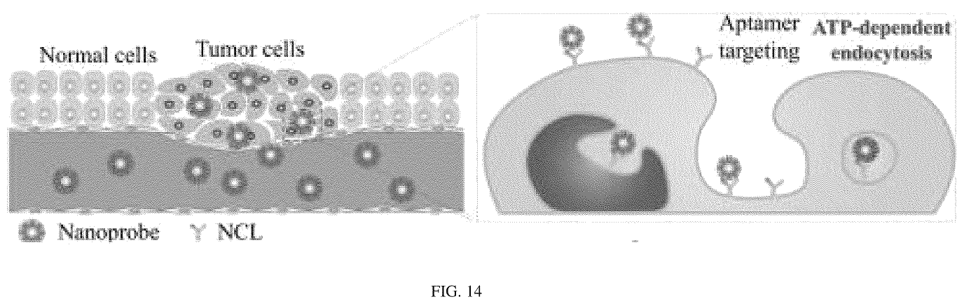

(4) Targeted identification of the NCL on the cell membrane surface was performed by the AS1411@RuPEP nanoprobe, and an assumed process of selectively imaging tumor cells was shown in FIG. 14.

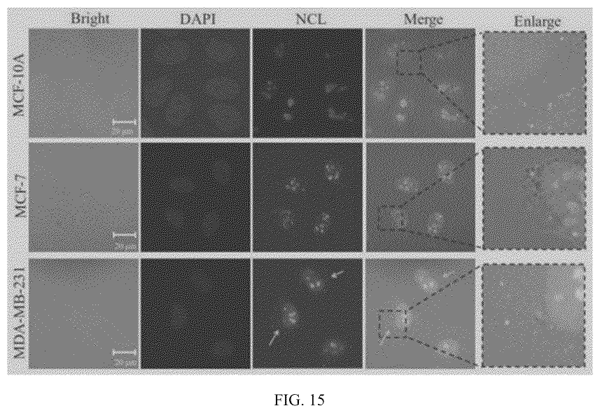

NCL was a major nucleolin, which could shuttle between a cell surface, cytoplasm and nucleus, which property made the NCL become an attractive target for selective delivery of an anti-tumor drug without affecting normal cells. Many studies had shown that, the NCL was overexpressed in human breast cancer cells and mainly distributed on the surface of the cell membrane. However, in normal epithelial cells, the NCL was mainly confined in the nucleus and scarce in the cell membrane. The distribution of the NCL in breast cancer MDA-MB-231, MCF-7 cells and normal human MCF-10A cells was shown in FIG. 15. It could be seen that in both cases, the NCL was clearly located in the nucleolus, which was perfectly matched with the DAPI-stained nucleus and unstained nucleolus, indicating that the NCL was mainly distributed in the nucleus, with distribution of only a small amount in the cell membrane and abundant distribution in tumor cells.

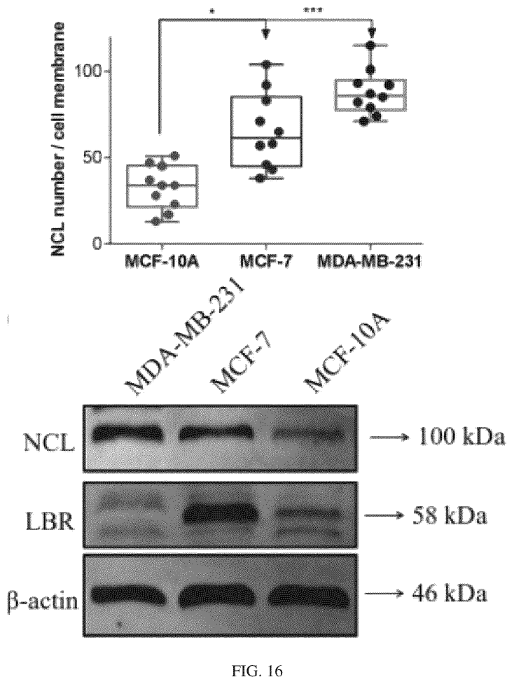

The expression of the NCL in breast cancer MDA-MB-231, MCF-7 cells and normal human MCF-10A cells was shown in FIG. 16. This showed that the expression of the NCL in the MDA-MB-231 cells was significantly higher than that in the MCF-10A cells.

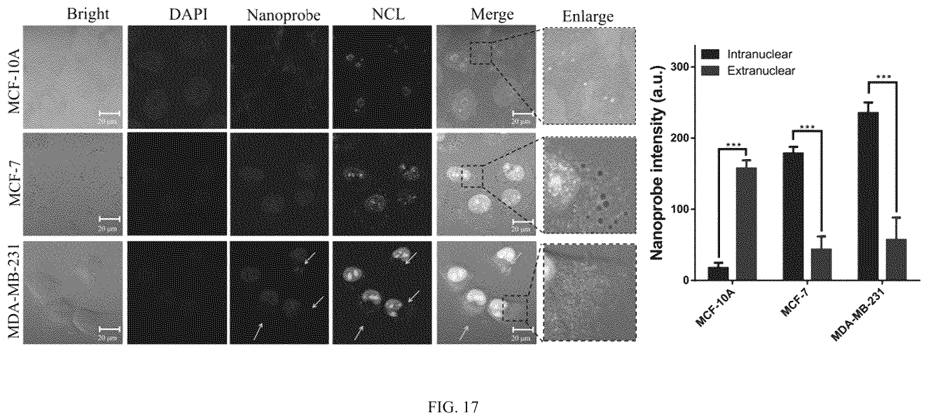

(5) The localization of the AS411@RuPEP nanoprobe in breast cancer MDA-MB-231, MCF-7 cells and normal human MCF-10A cells was shown in FIG. 17. As could be seen from FIG. 17, for the MCF-10A cells, the nanoprobe could not enter the cells, and the NCL targets in normal epithelial cells showed weak and diffuse uptake. However, for the MDA-MB-231 cells, in the presence of the nanoprobe, DAPI staining in the nucleus was overlapped with the probe color, and the number of NCL sites was significantly higher than that in the MCF-10A cells. These results indicated that the nanoprobe could selectively identify and activate the transportation of an NCL receptor on a surface of a cell to the nucleus, thereby facilitating the imaging of breast cancer cells.

(6) In order to further evaluate the selectivity of the nanoprobe to the breast cancer cells, a co-culture model of MDA-MB-231 and MCF-10A cells was established on the microscope slide in the present disclosure. Considering the overexpression of the NCL in the human breast cancer cell MDA-MB-231 and the lack of it in the normal immortalized human epidermal cell MCF-10A, it could be inferred that the uptake of the nanoprobe should be preferentially located in a breast cancer cell line. In order to distinguish the two cell lines in co-culture, green fluorescent MCF-10A cells labeled with GFP for actin were used, and all cells in the co-culture system were labeled with Hoechst 33258 blue fluorescence, and the co-culture cells were incubated with the nanoprobe at 5 μM for 6 hours.

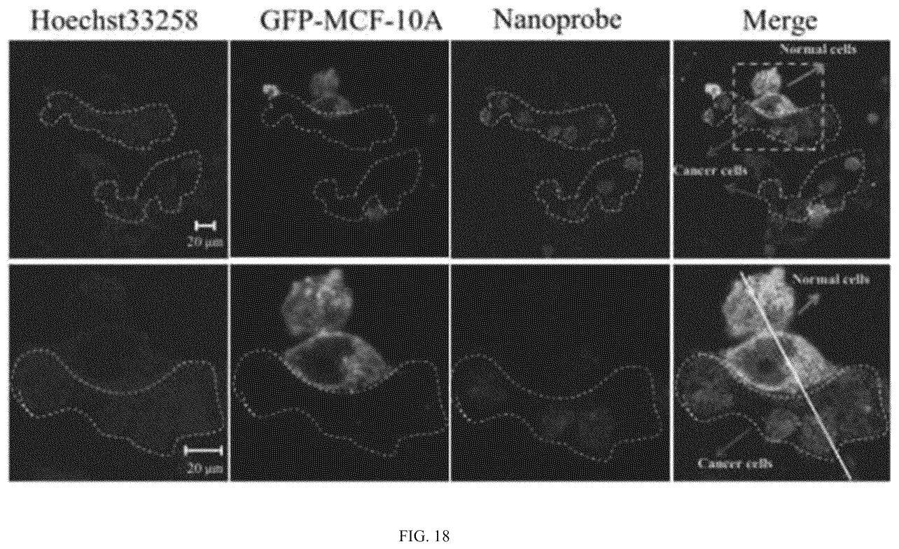

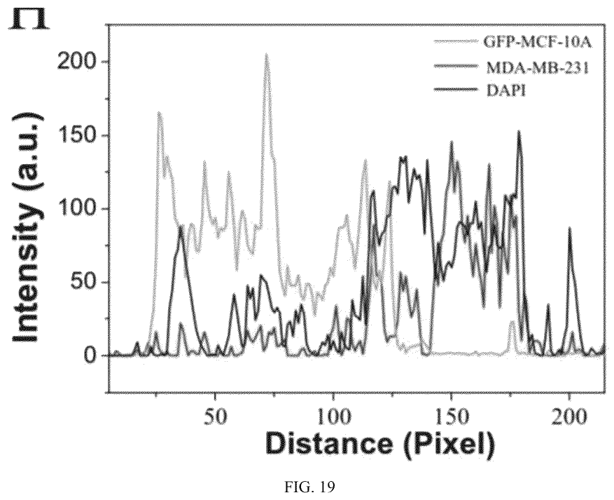

An LSCM image of the MDA-MB-231 and MCF-10A cells after being co-cultured in 0.2 mL of AS1411@RuPEP (5 μM) for 6 h was shown in FIG. 18. The overlay data was analyzed by using Image Pro Plus, and the obtained results were shown in FIG. 19. The results showed that there was intense red phosphorescence in the nucleuses of the MDA-MB-231 cells, but only weak red phosphorescence was observed in the MCF-10A cells. These results clearly showed that nanoprobe could specifically target and identify tumor cells in mixed culture.

### Example 3 In Vivo Imaging of Tumor Cells

Expression of AS1411@RuPEP in BALB/c mice bearing MDA-MB-231 tumors

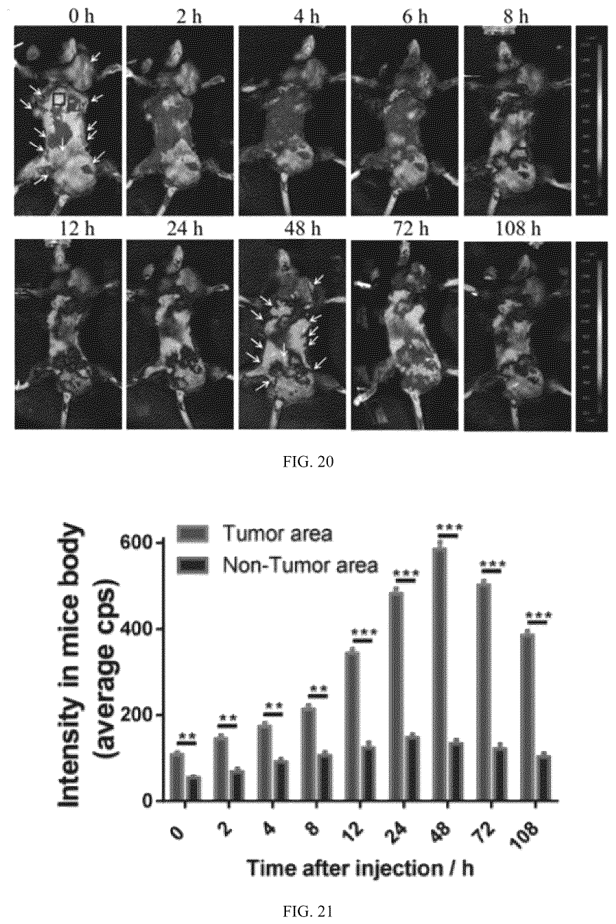

(1) 24-week-old female transgenic MMTV-PyMT mice with primary breast cancer (25-30 g) were purchased from Changzhou Cavens Experimental Animal Co., Ltd. 3 MMTV-PyMT mice with primary breast cancer were used as a control group, and intravenously injected with pure normal saline (100 μL) to evaluate the tumor-targeting effect and image quality of the nanoprobe at different time schemes in vivo. The other 3 mice were intravenously injected with an equivalent nanoprobe dose of 20 μM (100 μL). All animals were monitored by near infrared at 0, 2, 4, 6, 8, 12, 24, 48, 72 and 108 h. Tumor nodules and organs (heart, liver, spleen, lung, kidney and brain) were excised at 24, 72 and 108 h respectively, and in vitro near infrared imaging of them was performed. Specific tumor-targeted images were taken at different time points after injection of the nanoprobes, as shown in FIG. 20. The fluorescence intensity results of AS1411@RuPEP in a tumor region and a non-tumor region of the mouse by quantitative determination were shown in FIG. 21.

As could be seen from FIGS. 20-21, the near-infrared imaging of the mice before injection of the probe showed almost no signal. Due to the rapid distribution of the probe, the near-infrared phosphorescence could be seen at the tail of the mouse immediately after tail vein injection. Due to the enhanced permeability and retention effect (EPR effect), the nanoprobe could quickly identify and bind to an NCL target in a tumor tissue, so that it could quickly define the tumor region of the mouse within the first 6 hours. At 12 hours, due to the interference of a residual signal from a tumor site and fluorescent background from a normal tissue, the area of the highlighted tumor region was increased. Over time, the definition of the tumor region became not clear enough due to the interference from the fluorescence of the normal tissue, the clearance of the probe and nonspecific uptake.

In contrast, for the non-targeted probe RuPEP, intense phosphorescence was observed in vivo throughout the mouse within 6 hours, which indicated that the free RuPEP was rapidly distributed throughout the body and increased over time. The results showed that the nanoprobe of the present disclosure could selectively and rapidly locate the tumor tissue within 6 hours of systematic administration. Eventually, the nanoprobe would be distributed throughout the body, but still mainly accumulated in the tumor.

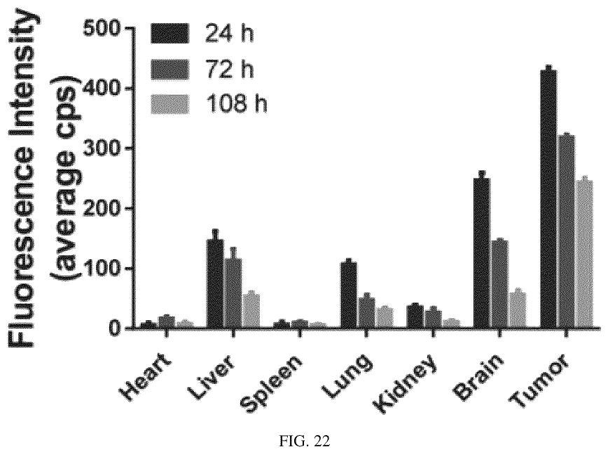

The fluorescence intensity result of AS1411@RuPEP (average cps) in an anatomical organ or tissue determined quantitatively was shown in FIG. 22, and in FIG. 22, *p<0.05, **p<0.01, and ***p<0.001. As could be seen from FIG. 22, the retention of the nanoprobe in the tumor at 24 hours and 48 hours was similar, indicating that the nanoprobe had a longer retention time in the tumor. In vitro images after dissection showed that the fluorescence intensity of different organs determined the quantitative distribution of the nanoprobe and the non-targeted RuPEP component. The signals of the two probes in a brain tissue at 24 hours were significantly higher than those at 48 hours, while the signals in kidney at 24 hours were significantly lower than those at 48 hours. This showed that both probes were transported through a blood-brain barrier and cleared away from the body by renal filtration.

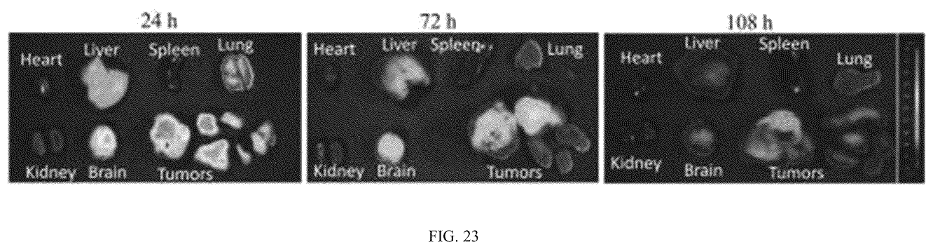

The tissue distribution and drug metabolism of AS1411@RuPEP at 24, 72 and 108 h were shown in FIG. 23. As could be seen from FIG. 23, low metabolism and slow renal clearance led to high aggregation of the probe in the tumor, which made the fluorescence intensity of the nanoprobe at 24 hours higher than that at 48 hours. Both in vivo measurement and in vitro measurement after organ removal showed that the aggregation of the probe in the tumor and kidney. Although the probe would be cleared away in the kidney, these data all consistently showed that it was feasible to use the probe for non-invasive real-time in vivo imaging of a specific tumor.

### Example 4 Preliminary Safety Evaluation In Vivo

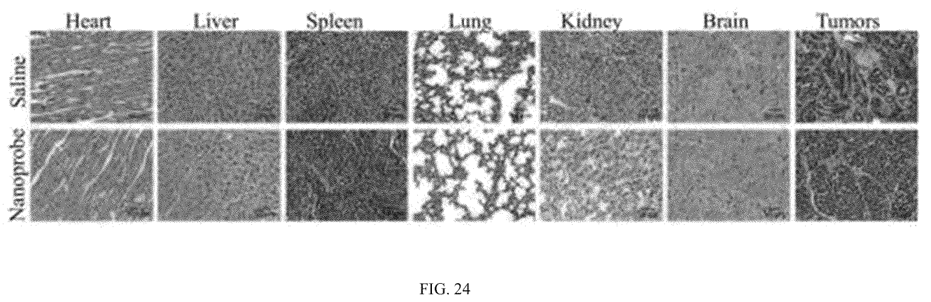

Healthy Kunming mice were used for evaluating the systemic toxicity of the nanoprobe. The nanoprobe was injected through tail vein at a dose of 50 mg/kg/day for 3 consecutive days. Then, primary tissues such as heart, liver, spleen, lung, kidney and brain were taken, subjected to HE staining, and observed for the histopathological changes under an optical microscope. The obtained results were shown in FIG. 24, with a scale of 50 μm. No death or serious weight loss was found in all experimental groups during the study. As could be seen from FIG. 24, all of the main tissues of the two groups, including heart, liver, spleen, lung and kidney, did not show obvious histopathological abnormalities and damage. These results showed that multiple doses of the nanoprobe had little effect on these tissues, indicating that the nanoprobe had not caused obvious side effects.

### Example 5 Potential Application of Nanoprobe as Diagnostic Reagent for Breast Cancer in Clinical Tissue Sample

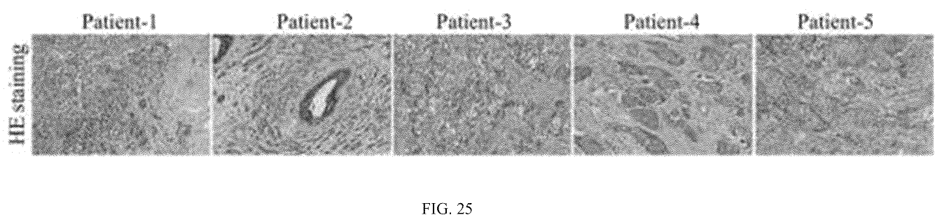

Fresh biopsy samples from 5 patients with breast invasive ductal carcinoma were used for evaluating the effectiveness of targeting the NCL by the nanoprobe in imaging of a tumor tissue. Histochemical analysis of hematoxylin-eosin staining of sections of the human breast cancer tissue from 5 patients with invasive ductal carcinoma was shown in FIG. 25. As could be seen from FIG. 25, the histological examination of the excised sample showed that an obvious tumor lesion consisted of large polygonal cells, which were arranged in infiltrating solid and micropapillary, with abundant cytoplasm and being acidophilic, vacuolated and foamy. The in-situ region of the lesion contained alveolar-like cells arranged in the appearance of shoe tacks. Furthermore, it could be clearly seen that most of the nuclei in the image were segmented, and there was almost no contour corresponding to a non-epithelial nuclear object. However, there was obvious difference between the tumor tissue and a paracancer tissue. It could be seen that the cells of the tumor tissue were arranged in disorder and had a loose structure, and the nucleoli of them were larger and stained darker than those of normal cells.

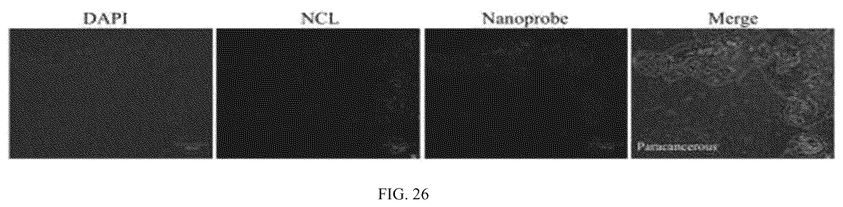

The imaging results of frozen sections of a fresh human invasive ductal carcinoma tissue observed under a fluorescence microscope were shown in FIG. 26. As could be seen from FIG. 26, in the histological analysis of in vitro tumor samples, blue phosphorescence in pathological sections could be observed under a DAPI channel. Furthermore, there was a clear boundary between a cancerous region with high expression of nucleolin (green fluorescent spots) and a paracancer region with low expression of nucleolin.

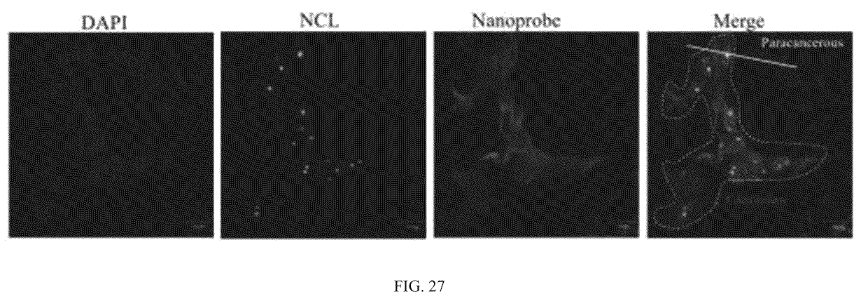

The distribution of the nucleolin and the nanoprobe in the cancer region and the paracancer region was observed by CLSM amplification, and the results were shown in FIG. 27. In FIG. 27, the whole tissue was stained with blue for DAPI, green for NCL and red for the nanoprobe. As could be seen from FIG. 27, the red phosphorescence of the nucleolin in the tumor region and the nucleus was largely overlapped, while there was no fluorescence signal of the nanoprobe and nucleolin in the paracancer region.

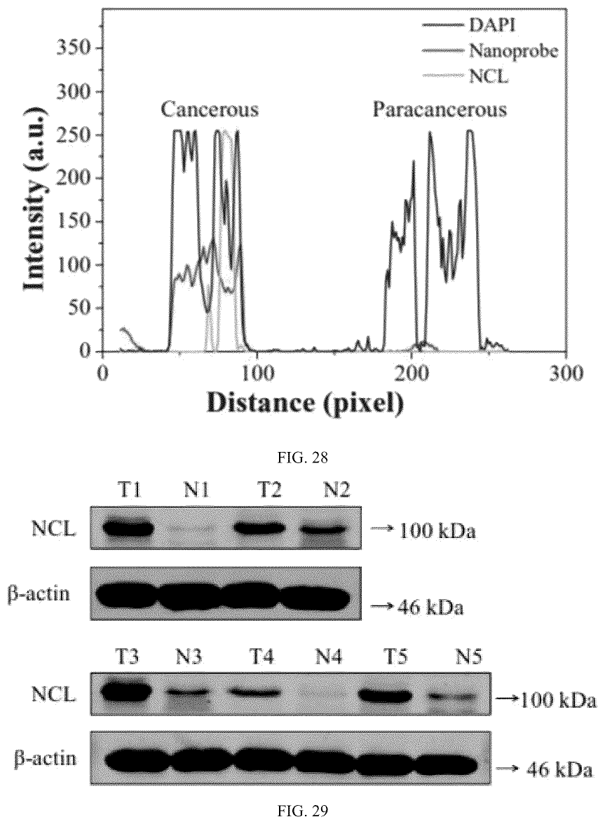

A combined curve of three-channel emission intensity of paracancer cells analyzed by Image-Pro Plus software was shown in FIG. 28. As could be seen from FIG. 28, the red phosphorescence of the nanoprobe perfectly merged with the green fluorescence of nucleolin in the cancer tissue, while there was no obvious red phosphorescence in the paracancer tissue.

The results showed that the AS1411@RuPEP nanoprobe of the present disclosure could effectively and distinctively highlight the cancer tissue in the biopsy sample of breast invasive ductal carcinoma.

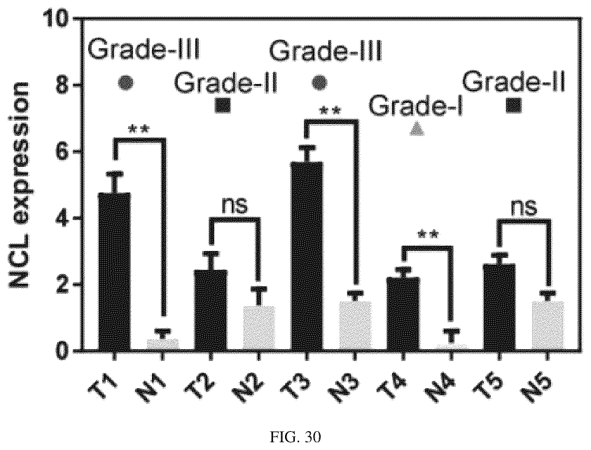

The expression of the NCL in a tumor tissue and a paracancer normal breast tissue was detected by Western Blotting. The expression of the NCL protein in paracancer tissues of 5 patients with invasive ductal carcinoma (n=5) was shown in FIG. 29. The quantitative analysis of NCL expression and tumor grading of the 5 cases of invasive ductal carcinoma was shown in FIG. 30. In FIG. 30, *P<0.05, **P<0.01, and ns represents non-significant. As could be seen from FIG. 29, the level of the NCL in most tumor tissues was significantly higher than that in adjacent normal tissues. As could be seen from FIG. 30, in conjunction with the results of a clinical diagnosis report, the high expression of the NCL indicated that the tumor had a high malignancy degree. This showed that the expression level of the NCL was a feasible defining feature of different malignant degrees of human invasive ductal carcinoma, which could be used for predicting the malignancy degree of tumor. By this method, the nanoprobe could be used for differentiating the malignancy degree of invasive ductal carcinoma in clinic.

### Example 6 Potential Clinical Application of Tumor Grading Diagnosis

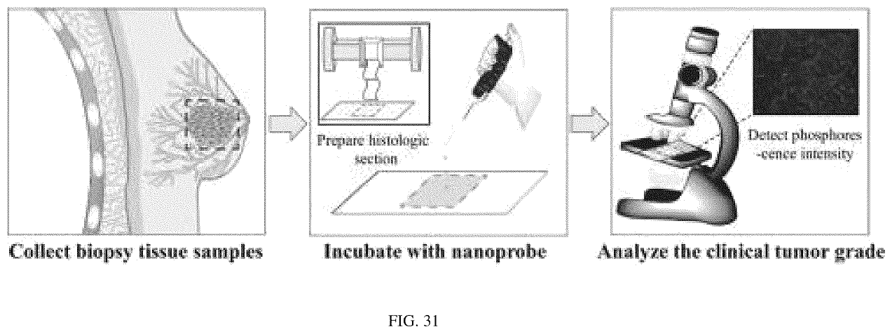

By detecting the luminous intensity in biopsy tissue sections, the feasibility of the nanoprobe as a convenient and rapid probe to determine tumor grading was evaluated. The operation flowchart of detecting a tumor tissue sample by the nanoprobe was shown in FIG. 31.

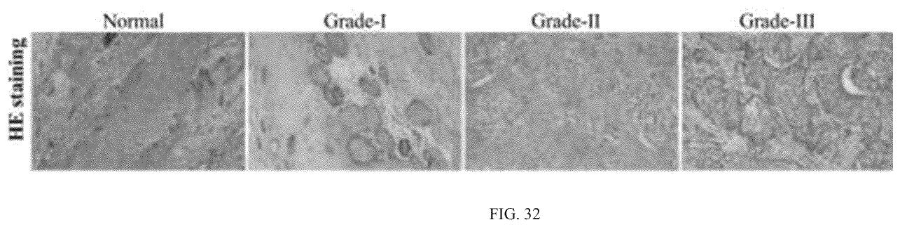

The pathological features of HE staining in normal and grade I-III tissues of invasive ductal carcinoma samples were shown in FIG. 32. As could be seen from FIG. 32, the HE staining results showed that normal tissue cells were densely arranged and reddish, and the grade I sample showed a clear tumor boundary and had no obvious invasion to adjacent normal tissues. However, with the development of the tumor to grades II and III, because of a large number of intertwined tumors, the boundary between malignant tumors and healthy tissues became blurred and eventually disappeared.

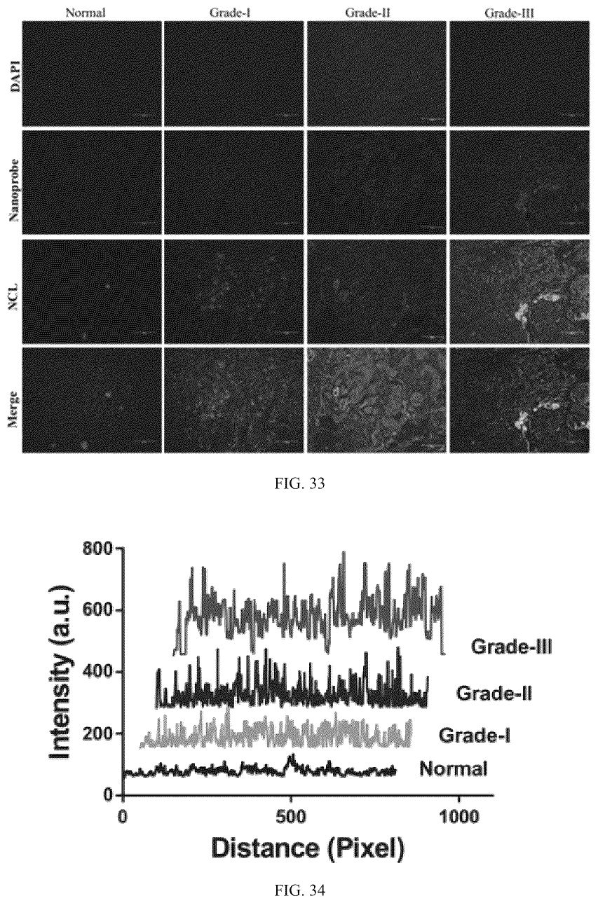

The results of imaging normal and different tumor-grading samples by 5 μM of the AS1411@RuPEP nanoprobe were shown in FIG. 33. As could be seen from FIG. 33, the imaging ability of the nanoprobe for different grades of invasive ductal carcinoma was significantly different. The higher the degree of malignancy was, the stronger the phosphorescence intensity was.

The emission intensity curves of the nanoprobe in different samples were shown in FIG. 34. As could be seen from FIG. 34, in these clinical samples, the nanoprobe emitted a very weak red signal in normal tissues, and the intensity of its labeling line was about 0-60 a.u., while quite strong red phosphorescence was observed in grade I-III tissues, and the intensity of its labeling line ranged from 110-260 a.u.

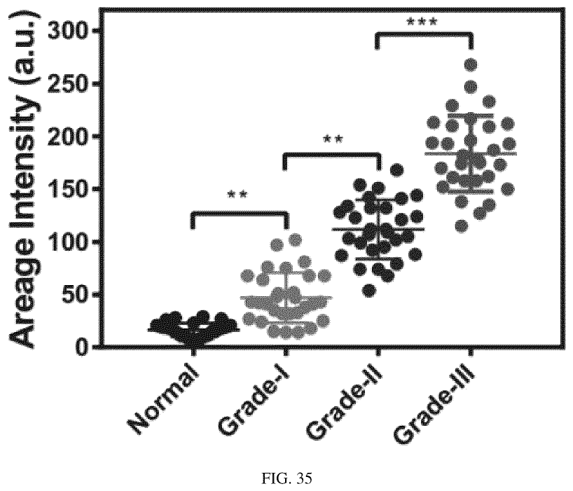

In order to further clarify the validity and reliability of the nanoprobe in differentiating tumor grades through a phosphorescence intensity range, it was necessary to expand the number of the samples and increase the number of duplicates. 10 normal samples of different tumor grades in each group were statistically analyzed for equal-area average intensity in 3 duplicates respectively, and the obtained results were shown in FIG. 35. In FIG. 35, n=10, *P<0.05, **P<0.01, and ns represented non-significant. By statistical analysis in 3 duplicate of 5 samples in each group, and it was found that the equal-area average intensity of the nanoprobe in a normal tissue was 7-21, 15-68 in a grade I tissues, 54-134 in a grade II tissue, and 88-152 in a grade III tissue.

In conclusion, in the present disclosure, in combination with the excellent tumor targeting ability of AS1411 and the strong phosphorescence emitting ability of the ruthenium complex (RuPEP), an AS1411@RuPEP nanoprobe was prepared, which could be used as a convenient and rapid tool to highlight and distinguish tumor cells in vivo and in vitro through targeted identification of the NCL. Moreover, the nanoprobe could further indicate the tumor grading and staging in pathological sections of a patient with breast cancer, which provided an effective way for clinical diagnosis and imaging of breast cancer.

The above description is only preferred embodiments of the present disclosure. It should be noted that, for those skilled in the art, several improvements and modifications can be made without departing from the principle of the present disclosure. These improvements and modifications should also be considered as falling within the protection scope of the present disclosure.

1. An AS1411 oligonucleotide composite ruthenium complex nanoprobe, comprising an AS1411 oligonucleotide having a G-quadruplex conformation and a ruthenium complex bound to the AS1411 oligonucleotide having the G-quadruplex conformation by hydrogen bonding, wherein the ruthenium complex has a structure of Formula 1:

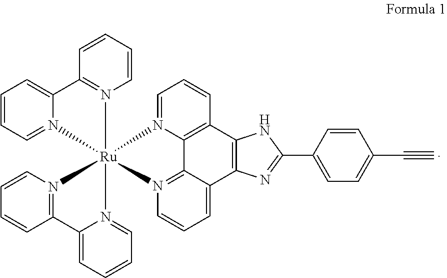

2. The AS1411 oligonucleotide composite ruthenium complex nanoprobe according to claim 1, wherein the content of ruthenium element in the AS1411 oligonucleotide composite ruthenium complex nanoprobe is 3-10 wt %.

3. The AS1411 oligonucleotide composite ruthenium complex nanoprobe according to claim 1, wherein the particle size of the AS1411 oligonucleotide composite ruthenium complex nanoprobe is 200-500 nm.

4. A preparation method of the AS1411 oligonucleotide composite ruthenium complex nanoprobe according to claim 1, comprising the following steps:
mixing a dispersion of an AS1411 oligonucleotide having a G-quadruplex conformation with a ruthenium complex having a structure of Formula 1, and performing self-assembly and dialysis, so as to obtain the AS1411 oligonucleotide composite ruthenium complex nanoprobe.

5. The preparation method according to claim 4, wherein the self-assembly is performed at a temperature of 25-40° C. for a period of 4-12 h.

6. The preparation method according to claim 4, wherein the molecular weight cutoff of the dialysis is 0.5-3.0 kDa; and the dialysis is performed at a temperature of 25-40° C. for a period of 1-3 days.

7. The preparation method according to claim 4, wherein a method for preparing the AS1411 oligonucleotide having the G-quadruplex conformation comprises the following steps:
mixing an AS1411 oligonucleotide with a buffer solution containing potassium ion, and sequentially performing high temperature denaturation and low temperature renaturation, so as to obtain the AS1411 oligonucleotide having the G-quadruplex conformation;
wherein the high temperature denaturation is performed at a temperature of 90-100° C. for a period of 5 min; and the low temperature renaturation is performed at a temperature of 4-8° C. for a period of 24-72 h.

8. Use of the AS1411 oligonucleotide composite ruthenium complex nanoprobe according to claim 1.

9. The use according to claim 8, wherein the cancer diagnostic reagent is a diagnostic reagent for breast cancer.

10. The AS1411 oligonucleotide composite ruthenium complex nanoprobe according to claim 2, wherein the particle size of the AS1411 oligonucleotide composite ruthenium complex nanoprobe is 200-500 nm.

11. The preparation method of claim 4, wherein the content of ruthenium element in the AS1411 oligonucleotide composite ruthenium complex nanoprobe is 3-10 wt %.

12. The preparation method of claim 4, wherein the particle size of the AS1411 oligonucleotide composite ruthenium complex nanoprobe is 200-500 nm.

13. The use of the AS1411 oligonucleotide composite ruthenium complex nanoprobe of claim 8, wherein the content of ruthenium element in the AS1411 oligonucleotide composite ruthenium complex nanoprobe is 3-10 wt %.

14. The use of the AS1411 oligonucleotide composite ruthenium complex nanoprobe of claim 8, wherein the particle size of the AS1411 oligonucleotide composite ruthenium complex nanoprobe is 200-500 nm.
